(19) United States
(12) Patent Application Publication
(10) Pub. No.: US 2021/0093469 A1
(43) Pub. Date: Apr. 1, 2021

(54) Cooling Cushion for Breast Form

(71) Applicant: American Breast Care, LP, Marietta, GA (US)

(72) Inventors: Robert Halley, Atlanta, GA (US); Amanda Wollnick, Doraville, GA (US); Leslie Amick, Marietta, GA (US)

(21) Appl. No.: 17/106,960

(22) Filed: Nov. 30, 2020

Related U.S. Application Data
(63) Continuation-in-part of application No. 16/408,676, filed on May 10, 2019, now Pat. No. 10,912,660.
Application No. 16/408,676 is a division of application No. 15/628,761, filed on Jun. 21, 2017, now Pat. No. 10,307,270.
(60) Provisional application No. 62/353,861, filed on Jun. 23, 2016.

(51) Int. Cl. A61F 2/52

(52) U.S. Cl. CPC A61F 2/52; A61F 2002/30088; A61F 2240/001

(57) ABSTRACT

A cooling cushion, for use in association with a breast prosthesis having a back surface worn by a user having a mastectomy region of a chest, includes cushion member that is conformable to both the mastectomy region of the chest of the user and the back surface of the breast prosthesis. A cooling material is disposed within the cushion member that moderates heat buildup in the mastectomy region of the chest of the user for at least a portion of time during which the user wears the cushion member. In making a cooling cushion, a suspension of soluble beads and an uncured silicone rubber gel is placed in a mold. The suspension is cured in the mold to generate a cooling cushion member. The cooling cushion member is removed after the curing step. The beads are dissolved from the cured cooling cushion member.

### CROSS-REFERENCE TO RELATED APPLICATION(S)

This application is a continuation-in-part of U.S. patent application Ser. No. 16/408,676, filed May 10, 2019, which is a divisional application of, and claims the benefit of, U.S. patent application Ser. No. 15/628,761, filed Jun. 21, 2017, now issued as U.S. Pat. No. 10,307,270, which claims the benefit of U.S. Provisional Patent Application Ser. No. 62/353,861, filed Jun. 23, 2016, the entirety of each of which is hereby incorporated herein by reference.

### BACKGROUND OF THE INVENTION

### 1. Field of the Invention

The present invention relates to breast prostheses and, more specifically, to a cooling insert for use with a breast prosthesis.

### 2. Description of the Related Art

A major complaint when wearing a breast prosthesis is heat being trapped behind the prosthesis leading to discomfort for the wearer. In traditional gel-type breast prostheses, several strategies have been developed to alleviate this problem. One such strategy involves adding a phase change material (PCM) to the gel. The PCM retains latent heat as it melts and, thereby maintains its temperature during its phase transition from solid phase to liquid phase. The PCM is chosen so as to have a melting point corresponding to the body temperature of the user so that temperature increase in the prosthesis is checked for a period after the user begins wearing it. However, gel-type breast prostheses require a film between the wearer and the gel. As a result, there is no direct contact between the user's chest and the PCM, thereby reducing the heat transfer efficiency of the prosthesis. Also, gel-type breast prostheses tend not to have a natural shape and do not lend themselves to the addition of desirable cosmetic features, such as veins and freckles, that result in a more natural look.

Custom rubber-type prostheses do have a natural shape and cosmetic features can be added to them. However, in custom rubber-type prostheses, heat buildup is a common concern for wearers. Such custom prostheses often employ a spongy material that acts as an insulator. As a result, wearing such prostheses can become uncomfortable after a limited amount of time.

Breast prostheses have traditionally consisted of 2-component silicone gel encased in polyurethane film. The two major sources of discomfort when wearing a breast prosthesis of this construction are the weight of the prosthesis and heat trapped behind the prosthesis. In traditional gel-type breast prostheses, lightweight fillers have been added to the silicone gel to alleviate the weight problem. To reduce the discomfort caused by heat, “vents” have been added to the shape of the prosthesis. These “vents” may not be successful. The gel/film construction of the prosthesis often does not produce a prosthesis firm enough to support a sustainable vent, and the “vents” collapse during normal wear. Another strategy for reducing the discomfort caused by heat is the addition of Phase Change Materials (PCM) to the silicone gel. In this construction, the effectiveness of the PCM is limited by its being trapped in a mass of insulating silicone.

Another construction of a breast prosthesis uses a “mechanical foam” silicone rubber. This type of foam allows for a more realistic feel than traditional foams, but it is not as life-like as silicone gels. This type of prostheses does have reduced weight but lacks the drape and response of silicone gel prostheses. Additionally, its construction can limit the amount of ventilation allowed by the mechanical foam if the skin layer is intimately contiguous with the patient's chest.

Therefore, there is a need for breathable cooling cushion for placement between a prosthesis and a user's chest.

### SUMMARY OF THE INVENTION

The disadvantages of the prior art are overcome by the present invention which, in one aspect, is a method of making a breast prosthesis for use by a wearer having a body temperature, in which a plurality of dissolvable beads is placed into an open back of a breast-shaped mold. The open back of the mold is sealed. A suspension of an uncured silicone rubber liquid and a plurality of phase change material pellets is injected into the mold around the beads. The uncured silicone rubber is allowed to cure, thereby forming a breast shape. The phase change material has a latent heat of fusion at a melting point so as to remove heat from the wearer when the body temperature is at least at the melting point. The breast shape is removed from the mold and the dissolvable beads are dissolved from the breast shape.

In another aspect, the invention is a breast prosthesis for use by a wearer who has a body temperature. A silicone rubber body portion has a shape corresponding to a human breast. The silicone rubber body portion defines a plurality of gas-filled voids therein. A phase change material is suspended within the silicone rubber of the body portion. The phase change material has a latent heat of fusion at a melting point so as to remove heat from the wearer when the body temperature is at least at the melting point.

In another aspect, the invention is an external breast prosthesis for use by a wearer who has a body temperature. A silicone rubber body portion has a shape corresponding to a human breast and defines a plurality of gas-filled voids therein. A paraffin phase change material is suspended within the silicone rubber. The paraffin phase change material has a predetermined number of carbon atoms per molecule so as to have a latent heat of fusion at a melting point so as to remove heat from the wearer when the body temperature is at least at the melting point. An outer skin layer is disposed about the silicone rubber body portion.

In another aspect, is a cushion and a breast prosthesis kit that combines the fit and feel properties of silicone gel prostheses with a mechanical foam cushion to alleviate the main issues of heat and weight which affect many breast prosthesis wearers. The invention includes a front component of traditional prostheses construction that includes a silicone gel encased in a plastic film sealed at the periphery of the front component; and a cushion including a mechanical silicone rubber foam configured to allow air flow between the front component and the wearer's chest.

In another aspect, the invention is a breathable cushion that can be applied to an existing breast prosthesis that includes a mechanical silicone rubber foam that is configured to allow air flow between the breast prosthesis and the wearer's chest.

In another aspect, the invention is a cooling cushion for use in association with a breast prosthesis having a back surface worn by a user having a mastectomy region of a chest. A cushion member is conformable to both the mastectomy region of the chest of the user and the back surface of the breast prosthesis. A cooling material is disposed within the cushion member that moderates heat buildup in the mastectomy region of the chest of the user for at least a portion of time during which the user wears the cushion member.

In another aspect, the invention is a breast prothesis cooling cushion for use in association with a breast prosthesis having a back surface worn by a user having a mastectomy region of a chest. A cushion member is conformable to both the mastectomy region of the chest of the user and the back surface of the breast prosthesis. The cushion member includes a mechanical open cell foam formed from a matrix defining a plurality of voids therein

In yet another aspect, the invention is a method of making a cooling cushion for use in association with a breast prosthesis having a back surface worn by a user having a mastectomy region of a chest. A suspension of a plurality of soluble beads and an uncured silicone rubber gel is placed in a mold having one surface corresponding to the back surface of the breast prosthesis. The suspension is cured while in the mold so as to generate a cured cooling cushion member. The cured cooling cushion member is removed after the curing step. The plurality of soluble beads is dissolved from the cured cooling cushion member.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram showing a side cross-sectional view of one embodiment of a breast prosthesis.

FIG. 2A-2E is a series of schematic diagrams demonstrating one method of making a breast prosthesis.

FIGS. 3A-3F is a series of schematic diagrams demonstrating a second method of making a breast prosthesis.

FIG. 4 is a schematic diagram showing a side cross-sectional view of one alternate embodiment of a breast prosthesis.

FIG. 5A is a side elevational view of an embodiment of a cushion and a prosthesis kit that are separated from each other.

FIG. 5B is a side elevational view of the embodiment shown in FIG. 1A in which the cushion and the prosthesis are placed next to each other.

FIG. 6 is an embodiment of a cushion and a prosthesis in which the cushion is secured to the prosthesis with a hook and loop fastener.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

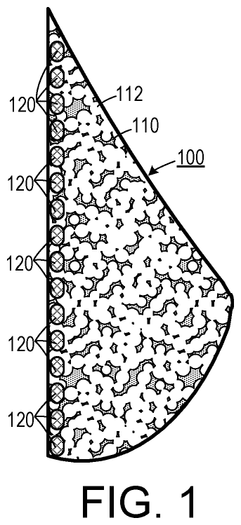

As shown in FIG. 1, one representative embodiment of a breast prosthesis 100 includes a rubber matrix 110 (that includes, for example, a silicone rubber) that suspends a plurality of air voids 112, or other lightweight inclusions, in the shape of a human breast.

A phase change material (PCM) is embedded in the rubber matrix 110. In one embodiment, the PCM can be in the form of pellets or capsules. In one representative embodiment, the PCM can include paraffin in which the average molecule has 20 or fewer carbon atoms. A paraffin with 20 carbon atoms per molecule would have a melting point of about 98° F. (which is the average body temperature of a person), whereas a paraffin with 14 carbon atoms per molecule would have a melting point of about 42° F. Thus, the melting point can be tuned to a specific desired temperature. In certain embodiments, PCMs of a range of melting points can be used so that they begin absorbing heat at different temperatures.

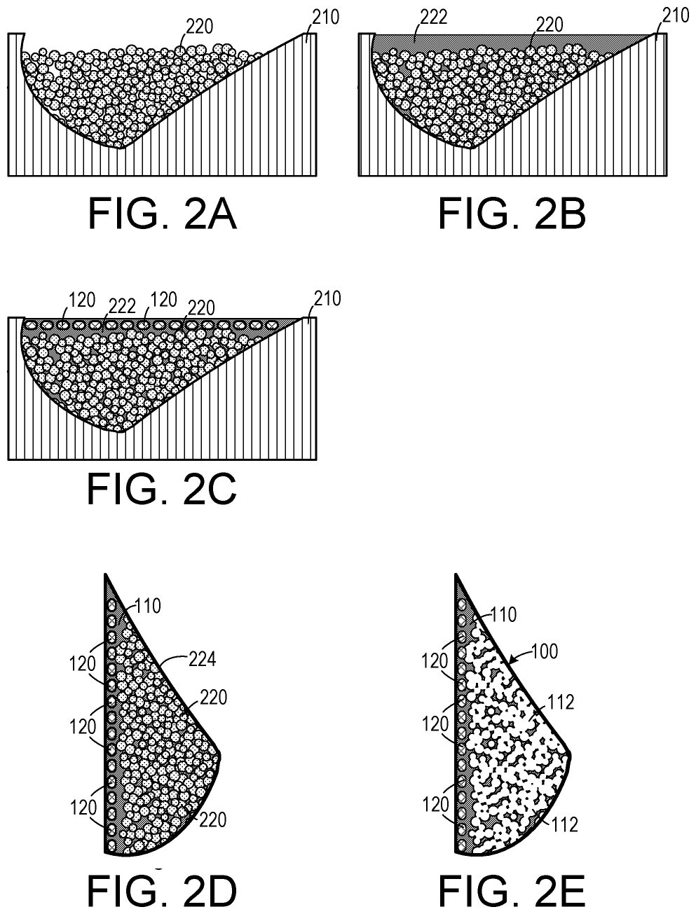

As shown in FIG. 2A, in one method of making a breast prosthesis, a plurality of beads 220 are poured into a mold 210 having a shape complimentary to that of a human breast. The beads could include a water-soluble substance such as common table sugar or salt. (In an alternate embodiment, the beads could include a lightweight non-soluble substance, such as expanded microspheres.) As shown in FIG. 2B, an uncured silicone rubber 222 is added to the beads 220. As shown in FIG. 2C, PCM pellets or capsules 120 are added to the uncured silicone rubber 222. The uncured silicone rubber 222 is cured and the cured breast form 224 is removed from the mold 210, as shown in FIG. 2D. As shown in FIG. 2E, the beads 220 are dissolved away from the breast form 224, leaving air voids 112 in the cured rubber matrix 110, resulting in the final breast prosthesis 100.

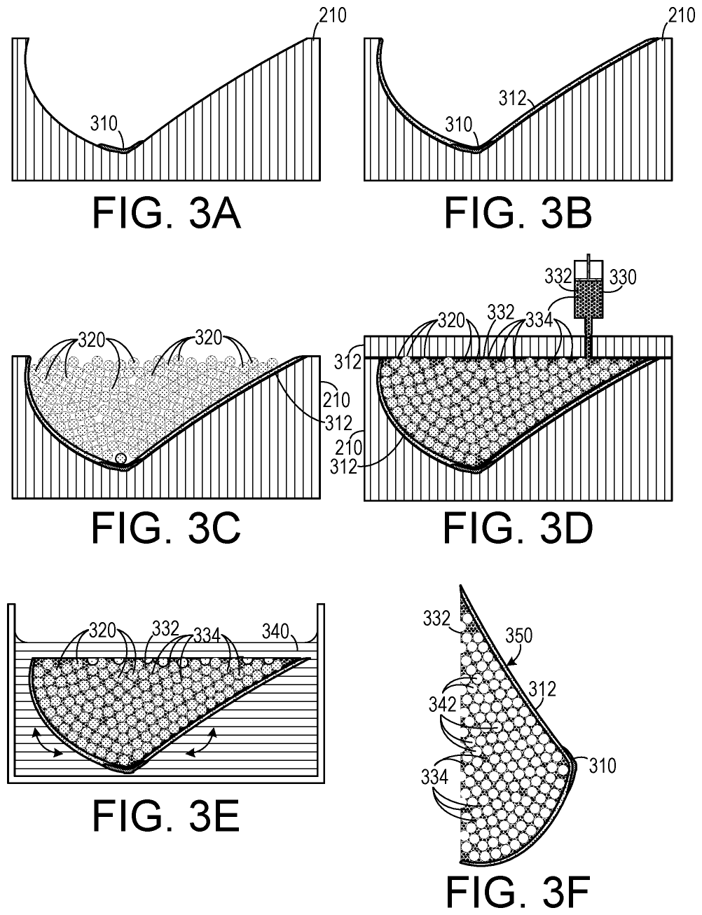

As shown in FIGS. 3A-3F, in one method of making a breast prosthesis, a liquid silicone rubber precursor that includes a pigment having the color of a natural nipple is applied to a portion of a mold 210 corresponding to the location of a nipple in the resulting prosthesis and is allowed to cure, thereby generating an image of a nipple 310. Once cured, a front skin layer 312 (or several layers in some embodiments) is applied to the mold and allowed to cure. The thickness of the layer(s) of silicone rubber and the firmness of the silicone rubber are chosen to have a feel similar to human skin. The skin layer 312 can have pigments and other additives to mimic the appearance and texture of skin. The skin layer 312 is formed to the shape of a breast by the mold 210, which can be either a custom shape from an individual measurement or a more generic shaped breast. Additional features, such as images of veins and freckles can be included in the skin layer 312.

Dissolvable beads 320 (such as sugar beads or salt beads) are placed into the mold 210 and a back 312 is affixed to the mold 210. In one embodiment, sugar beads (also referred to as “sugar pellets” and “sugar pearls”) of the type commonly known to the baking and pharmaceutical industries are used. In one embodiment, the sugar beads have a diameter of about 2 mm, in other embodiments they range in diameter from 1 mm to 4 mm. It has been found that sugar beads tend to dissolve more quickly than salt beads.

A suspension of silicon liquid rubber precursor 332 and phase change material pellets 334 (such a paraffin pellets) is injected into the mold 210 around the dissolvable beads 320 with a viscous liquid injecting device, such as an air gun-type viscous liquid pump. (The silicone rubber precursors for the nipple portion, the skin layer and the suspension can include, for example, Dragon Skin series or Ecoflex series two-part silicon available from Smooth-On, Inc., 5600 Lower Macungie Road, Macungie, Pa. 18062.) The silicone curing time typically varies between 30 minutes to four hours, depending upon the specific silicone formulation used. Typically, the silicone for nipple 310 and skin layer 312 is harder than fill silicone 332.

Once the silicone rubber 332 has cured, then the prosthesis 350 is placed in a solvent 340 (which could include water) to allow the dissolvable beads 320 to dissolve from the prosthesis, leaving air-filled voids 342 surrounded by a matrix of silicone rubber 332 and phase change material pellets 334. The dissolution can be helped by gently agitating and squeezing the prosthesis 350 during the dissolution period. The dissolution time depends upon the size of the prosthesis and typically takes between 10 minutes to several days to complete.

In most embodiments, the front of the mold 210 will have the shape of a breast as it would be held in a bra cup. The back 312 of the mold will have the shape of a chest. The shape can be produced to match a specific patient or can be a general shape. The mold 210 can be made of any typical mold making material including aluminum or tooling board.

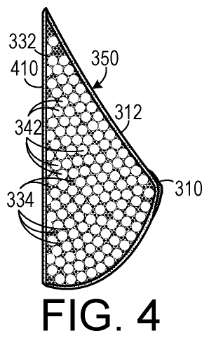

As shorn in FIG. 4, a back skin layer 410 may be applied to part or all of the back of the prosthesis 350. The skin layer 410 can also include phase change material pellets.

In one embodiment, phase change material pellets 334 can include pellets having different latent heats of fusion (and correspondingly different melting points) so that they sequester heat at different temperatures. For example, some of the pellets could be chosen to begin melting at 95° F., while others would begin melting at 85° F. and still others would begin melting at 75° F., which would allow for heat sequestration in various temperatures. (The typical body temperature of a wearer would be about 97.9° F., so this temperature range would provide rapid cooling as the 75° F. beads melted, followed by gradual cooling as the other beads melted.) In one embodiment, a phase change material with a transition temperature substantially below body temp can be used. For example, in an embodiment employing a phase change material with a transition temperature of 75° F., the prosthesis could be cooled in a refrigerator prior to being worn. The lower transition temperature would allow for the form to remain at a cooler temperature, which could be useful for a patient with sensitive or damaged skin. Also, the silicon rubber matrix material can be added in layers, which could contain different phase change materials (e.g., phase change materials that melt at different temperatures and also phase change material pellets of different sizes).

In one embodiment, dissolvable beads are not used, and the silicone rubber liquid includes chemical foaming agents, which result in the formation of gas-filled voids in the silicone rubber of the prosthesis as the silicone rubber cures.

The silicone rubber with PCM and gaseous voids of the present invention has several advantages over existing systems. For example, the present invention has an advantage over gel systems in that no film is necessary to contain a gel so that the PCM will be in material that can be directly touching the patient's chest, thereby improving heat transfer. The silicone rubber used creates a more realistic looking prosthesis, which can include the possibility of such cosmetic features as the images of nipple, veins, freckles, etc. The silicone rubber employed allows for more complex shapes. The silicone rubber allows for lighter breast prostheses than gel prostheses. Gel prostheses can have reduced densities in the 0.55 to 0.70 g/ml range. The present invention, on the other hand, can product a prostheses with a density of approx. 0.40 g/ml. This results in a significantly more comfortable wearing experience.

Methods for making porous silicone mechanical foam and silicone mechanical foam including phase change material are disclosed in U.S. Pat. No. 10,307,270, issued to Halley et al., which is hereby incorporated by reference for the purpose of disclosing such methods.

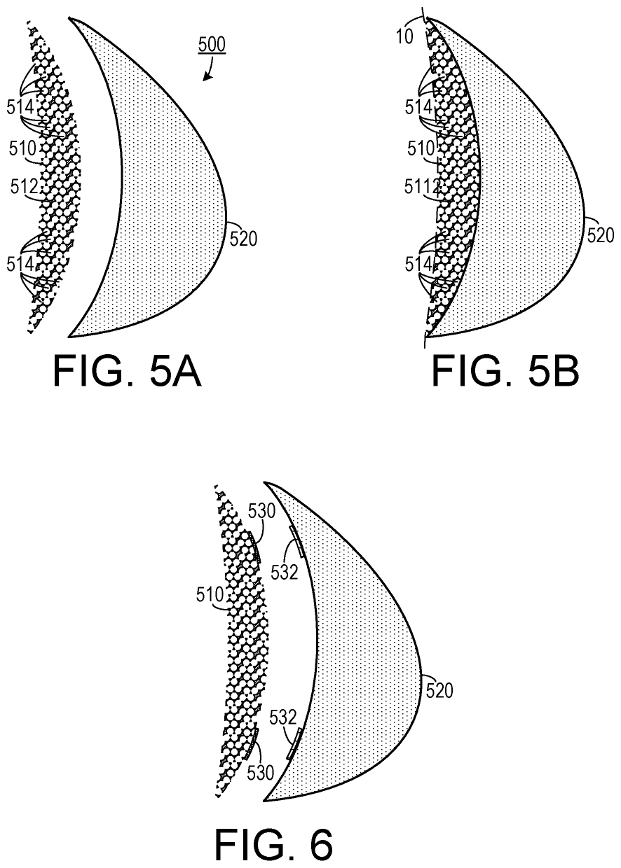

As shown in FIGS. 5A and 5B, in one embodiment of a cushion 510 for use with a prosthesis 520, the cushion 510 includes a silicone rubber matrix 512 that defines a plurality of voids 514 so as to form an open cell foam cushion. The cushion 510 is configured to conform to the user's chest 10 and to the back surface of the prosthesis 520. A phase change material (such as a paraffin powder) may be added to the silicone rubber matrix 512 to provide cooling to the wearer.

As shown in FIG. 6, the cushion 510 can be held to the prosthesis 520, in one example, using a fastener such as a hook and loop fastener. Many other ways of holding the cushion 510 in place may also be used. The fastener can include one or more tabs of a first type of hook and loop fastener material 530 affixed to the cushion member 520 and one tabs of a second type of hook and loop fastener material 532. If the first type is hook material, then the second type would be loop material and vise-versa.

The front component prosthesis 520 can include two plastic films welded at the periphery. Silicone gel is contained within the plastic films. The film and the gel are thermally formed so the front of the component is shaped like a breast and the back of the component is configured to accept the cool cushion 510 placed between it and the user's chest. The front component can be made by various techniques know in the art, such as is disclosed in U.S. Pat. No. 8,562,679, issued to Rechenberg, which is hereby incorporated by reference. An alternate embodiment includes a cushion 510 that is adapted for use with conventional prostheses.

In the front component prosthesis 520, the gel can be firmer or softer depending on the desired shape, drape and target customer's body type. The gel can contain pigments and other additives to make the appearance more realistic or pleasing. The gel can contain weight reducing fillers such as Expancel Microspheres. The film is preferably polyurethane but can be any thermoplastic film of sufficient strength and elasticity. The film can be pigmented of modified with printing via techniques well known in the art. The back of the front component can be substantially flat and either friction or an adhesive can be used to hold the cushion in place. The back of the front component can be shaped to accept the cool cushion of mechanical foam. The shape can be modified to accept the cushion in a lock and key arrangement or fasteners such as hook and loop, magnets or snaps can be added to the back of the front component to accept similar fasteners affixed to the cool cushion.

In one embodiment, the cool cushion 510 is made of mechanical foam of silicone rubber. Two-component silicone rubber is particularly suited to the invention. The two-component mixture is easily mixed and poured into various shapes prior to cure. Silicone is safe and comfortable against the skin

In one embodiment, the mechanical foam includes 45%-75% voids by volume and is of an open cell construction. The voids 514 are connected to allow multiple routes for air flow. The mechanical foam is comfortable enough to wear against the skin, but its structure is sturdy enough to hold its shape under typical wearing conditions. Other rubbers such as latex rubber, thermoplastic elastomer or polyurethane rubber can also be used. Similarly, typical open-cell, chemical foam structures may not work in this application as they tend not to have an appropriate cell structure or are not comfortable for wearing.

The mechanical foam cushion 510 can include pores of different sizes. The pore sizes are determined by the mechanical foaming agent being used. The rubber used in the cushion can be of different hardnesses. The rubber is typically firm enough to support the voids for air flow, but also soft enough that it is comfortable to wear. The rubber used in the cushion can have different additives such as pigment, etc. typically used in silicone rubbers. The PCM can be designed to go through phase transitions in the range of human skin or body temperatures to further mitigate the effects of heat while wearing the cushion. The air flow enabled by the mechanical foam will allow the PCM to be a more effective heat mitigation additive. The air flow will move heat through the cushion more effectively. The PCM can be a mixture of PCM with various phase transition temperatures. For example, PCM's with transition temperature of 95 F, 85 F and 75 F can be combined. The PCM can be a pure material or it can be an encapsulated PCM material. The PCM can be wax or other typical material used for PCMs.

The front of the cushion 510 can be flat so that it is affixed to the front component via friction during use. The front of the cushion 510 can be shaped so that it is affixed to the front component via lock and key arrangement with appropriate shapes on the back of the front component. The front of the cushion 510 can have a tacky gel or reusable adhesive that will affix it to the back of the front component. The front of the cushion 510 can use pressure sensitive adhesive tape or liquid adhesive to affix the cushion to the back of the front component. The cushion 510 can be permanently affixed to the front component via adhesive or solvent welding. The rear of the cushion 510 can be shaped to have protuberances or recession as desired to aid in comfort or airflow.

The following are examples of methods of producing a cushion 510:

In a first example:
A mold of the desired shape is produced with appropriate venting.
The mold is charged with sugar (or salt) beads of approx. 2 mm diameter. The mold is filled to capacity.
An uncured silicone rubber solution is pumped into the molds around the sugar beads.
The cured part is removed from the mold.
The sugar beads are dissolved out of the silicone rubber.
The silicone rubber cushion is dried.

In a second example:
A mold of the desired shape is produced with appropriate venting.
The mold is charged with sugar (or salt) beads of approx. 2 mm diameter. The mold is filled to capacity.
An uncured silicone rubber solution containing 10% by weight of an encapsulated PCM with a phase change temperature of 32° C. is pumped into the molds around the sugar beads.
The cured part is removed from the mold.
The sugar beads are dissolved out of the silicone rubber.
The silicone rubber cushion containing PCM is dried.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A cooling cushion for use in association with a breast prosthesis having a back surface worn by a user having a mastectomy region of a chest, comprising:
(a) a cushion member that is conformable to both the mastectomy region of the chest of the user and the back surface of the breast prosthesis; and
(b) a cooling material disposed within the cushion member that moderates heat buildup in the mastectomy region of the chest of the user for at least a portion of time during which the user wears the cushion member.

2. The cooling cushion of claim 1, wherein the cooling material comprises a phase change material that absorbs heat while transitioning from a first phase to a different second phase.

3. The cooling cushion of claim 2, wherein the phase change material comprises a paraffin.

4. The cooling cushion of claim 3, wherein average molecules of the paraffin include 20 or fewer carbon atoms.

5. The cooling cushion of claim 1, wherein the cushion member comprises a mechanical foam.

6. The cooling cushion of claim 5, wherein the mechanical foam comprises a silicon rubber matrix that defines a plurality of voids.

7. The cooling cushion of claim 6, wherein in the voids are in fluid communication with each other so that the mechanical foam is an open cell foam.

8. The cooling cushion of claim 1, further comprising a fastening mechanism for holding the cushion member in a fixed relationship with the breast prosthesis.

9. The cooling cushion of claim 8, wherein the fastening mechanism comprises:
(a) a first type of hook and loop fastener material affixed to the cushion member; and
(b) a second type of hook and loop fastener material that engages with the first type of hook and loop fastener material that is affixed to the back surface of the breast prosthesis and that is disposed in alignment with the first type of hook and loop fastener material.

10. A breast prothesis cooling cushion for use in association with a breast prosthesis having a back surface worn by a user having a mastectomy region of a chest, comprising a cushion member that is conformable to both the mastectomy region of the chest of the user and the back surface of the breast prosthesis, the cushion member including a mechanical open cell foam formed from a matrix defining a plurality of voids therein.

11. The breast prothesis cooling cushion of claim 10, further comprising a cooling material disposed within the cushion member that moderates heat buildup in the mastectomy region of the chest of the user for at least a portion of time during which the user wears the cushion member, the cooling material comprising a phase change material that absorbs heat while transitioning from a first phase to a different second phase, wherein the phase change material comprises a paraffin in which average molecules of the paraffin include 20 or fewer carbon atoms.

12. The breast prothesis cooling cushion of claim 10, wherein the matrix comprises a silicon rubber and wherein in the voids are in fluid communication with each other so that the mechanical foam is an open cell foam.

13. The breast prothesis cooling cushion of claim 10, further comprising a fastening mechanism for holding the cushion member in a fixed relationship with the breast prosthesis, the fastening mechanism comprising:
(a) a first type of hook and loop fastener material affixed to the cushion member; and
(b) a second type of hook and loop fastener material that engages with the first type of hook and loop fastener material that is affixed to the back surface of the breast prosthesis and that is disposed in alignment with the first type of hook and loop fastener material.

14. A method of making a cooling cushion for use in association with a breast prosthesis having a back surface worn by a user having a mastectomy region of a chest, comprising the steps of:
(a) placing a suspension of a plurality of soluble beads and an uncured silicone rubber gel in a mold having one surface corresponding to the back surface of the breast prosthesis;
(b) curing the suspension while in the mold so as to generate a cured cooling cushion member;
(c) removing the cured cooling cushion member after the curing step;
(d) dissolving the plurality of soluble beads from the cured cooling cushion member.

15. The method of claim 14, wherein the soluble beads comprises a selected one of sugar beads and salt beads, and wherein the step of dissolving the plurality of soluble beads comprises placing the cured cooling cushion member in water.

16. The method of claim 14, further comprising the step of suspending a phase change material powder to the uncured silicone rubber gel.

17. The method of claim 16, wherein the phase change material comprises a paraffin.

18. The method of claim 14, further comprising the step of selecting the paraffin so that average molecules of the paraffin include 20 or fewer carbon atoms.

19. The method of claim 14, further comprising the step of installing a fastening mechanism for holding the cushion member in a fixed relationship with the breast prosthesis.

20. The method of claim 19, wherein the step of installing a fastening mechanism comprises the steps of:
(a) affixing a first type of hook and loop fastener material to the cushion member; and
(b) affixing a second type of hook and loop fastener material that engages with the first type of hook and loop fastener material to the back surface of the breast prosthesis so as to be in alignment with the first type of hook and loop fastener material.
